(19) United States
(12) Patent Application Publication
(10) Pub. No.: US 2020/0002468 A1
(43) Pub. Date: Jan. 2, 2020

(54) Compound and Organic Solar Cell Comprising Same

(71) Applicant: LG Chem, Ltd., Seoul (KR)

(72) Inventors: Bogyu Lim, Daejeon (KR); Songrim Jang, Daejeon (KR); Doowhan Choi, Daejeon (KR); Ji Hoon Kim, Daejeon (KR)

(73) Assignee: LG Chem, Ltd., Seoul (KR)

(21) Appl. No.: 16/482,021

(22) PCT Filed: Jun. 4, 2018
(86) PCT No.: PCT/KR2018/006358

§ 371 Date: Jul. 30, 2019

(30) Foreign Application Priority Data
Jun. 8, 2017 (KR) 10-2017-0071665

(51) Int. Cl. C08G 61/12; H01L 51/00; C07D 495/04

(52) U.S. Cl. CPC C08G 61/126; H01L 51/4273; H01L 51/0036; C07D 495/04; H01L 51/0071; H01L 51/0074; H01L 51/0068; C08G 2261/91; C08G 2261/3246; C08G 2261/3243; C08G 2261/3223; C08G 2261/228; C08G 2261/18; C08G 2261/149; C08G 2261/1412; C08G 2261/1428; C08G 2261/124; C08G 2261/1424; C08G 2261/146; H01L 51/0043

(57) ABSTRACT

The present specification provides a compound including a unit of Formula 1 and an organic solar cell including the same.

### CROSS-REFERENCE TO RELATED APPLICATIONS

The present application is a national phase entry under 35 U.S.C. § 371 of International Application No. PCT/KR2018/006358 filed Jun. 4, 2018, which claims priority from Korean Patent Application No. 10-2017-0071665 filed Jun. 8, 2017, all of which are incorporated herein by reference.

### TECHNICAL FIELD

The present specification relates to a compound and an organic solar cell including the same.

### BACKGROUND ART

An organic solar cell is a device which may directly convert solar energy into electric energy by applying a photovoltaic effect. A solar cell may be divided into an inorganic solar cell and an organic solar cell, depending on the materials constituting a thin film. Typical solar cells are made through a p-n junction by doping crystalline silicon (Si), which is an inorganic semiconductor. Electrons and holes generated by absorbing light diffuse to p-n junction points and move to an electrode while being accelerated by the electric field. The power conversion efficiency in this process is defined as the ratio of electric power given to an external circuit and solar power entering the solar cell, and the efficiency have reached approximately 24% when measured under a currently standardized virtual solar irradiation condition. However, since inorganic solar cells in the related art have already shown the limitation in economic feasibility and material demands and supplies, an organic semiconductor solar cell, which is easily processed and inexpensive and has various functionalities, has come into the spotlight as a long-term alternative energy source.

For the solar cell, it is important to increase efficiency so as to output as much electric energy as possible from solar energy. In order to increase the efficiency of the solar cell, it is important to generate as many excitons as possible inside a semiconductor, but it is also important to pull the generated charges to the outside without loss. One of the reasons for the charge loss is the dissipation of generated electrons and holes due to recombination. Various methods have been proposed to deliver generated electrons and holes to an electrode without loss, but additional processes are required in most cases, and accordingly, manufacturing costs may be increased.

### DETAILED DESCRIPTION OF THE INVENTION

### Technical Problem

An object of the present specification is to provide a compound and an organic solar cell including the same.

### Technical Solution

The present specification provides a compound including a unit of the following Formula 1.

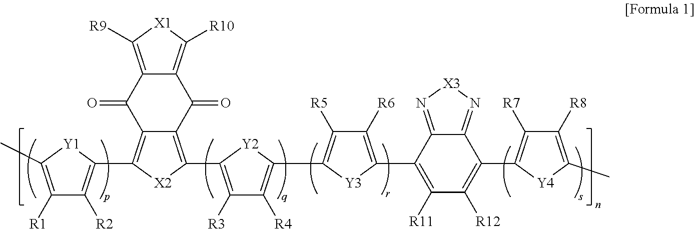

In Formula 1,

p and q are the same as or different from each other, and are each independently an integer from 0 to 3,

when p and q are each 2 or more, the structures in the parenthesis are the same as or different from each other,

r and s are the same as or different from each other, and are each independently an integer from 1 to 3,

when r and s are each 2 or more, the structures in the parenthesis are the same as or different from each other,

X1 to X3 are the same as or different from each other, and are each independently S, O, Se, Te, NR, CRR′, SiRR′, PR, or GeRR′,

Y1 to Y4 are the same as or different from each other, and are each independently S, O, Se, Te, NR, CRR′, SiRR′, PR, or GeRR′,

R1 to R12, R, and R′ are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heterocyclic group, and

n is an integer from 1 to 10,000.

Another exemplary embodiment of the present specification provides an organic solar cell including: a first electrode;

a second electrode provided to face the first electrode; and

an organic material layer having one or more layers provided between the first electrode and the second electrode and including a photoactive layer,

in which one or more layers of the organic material layer include the compound.

### Advantageous Effects

A compound according to an exemplary embodiment of the present specification exhibits planarity and thus has excellent aggregation characteristics and crystallinity.

The compound according to an exemplary embodiment of the present specification may have effects of decreasing a band gap and/or increasing an amount of light absorbed. Accordingly, as the compound according to an exemplary embodiment of the present specification exhibits a high current value (Isc) when applied to an organic solar cell, the compound may exhibit excellent efficiency.

The compound according to an exemplary embodiment of the present specification implements high efficiency and simultaneously has an appropriate solubility, and thus has an economic advantage in terms of time and/or costs during the manufacture of a device.

The compound according to an exemplary embodiment of the present specification may be used either alone or in mixture with other materials in an organic solar cell.

### BRIEF DESCRIPTION OF DRAWINGS

FIG. 1 is a view illustrating an organic solar cell according to an exemplary embodiment of the present specification:
101: Substrate
102: First electrode
103: Hole transport layer
104: Photoactive layer
105: Second electrode

FIG. 2 is a view illustrating an MS measurement result of Compound C.

FIG. 3 is a view illustrating an NMR measurement result of Compound C.

FIG. 4 is a view illustrating an MS measurement result of Compound D.

FIG. 5 is a view illustrating an NMR measurement result of Compound D.

FIG. 6 is a view illustrating photoelectric conversion characteristics of organic solar cells according to exemplary embodiments of the present specification.

### BEST MODE

Hereinafter, the present specification will be described in detail.

An exemplary embodiment of the present specification provides the compound represented by Formula 1.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

Examples of the substituents in the present specification will be described below, but are not limited thereto.

In the present specification,

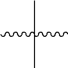

means a site bonded to another substituent, a monomer, or a binding portion.

In the present specification, “unit” means a repeated structure included in a compound. That is, “unit” may mean a structure included in the form of a divalent group or more in a compound by a polymerization reaction.

In the present specification, the meaning of “including a unit” means that the unit is included in a main chain in a compound.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, the term “substituted or unsubstituted” means being substituted with one or two or more substituents selected from the group consisting of deuterium; a halogen group; a nitrile group; a nitro group; an imide group; an amide group; a carbonyl group; an ester group; a hydroxyl group; an alkyl group; a cycloalkyl group; an alkoxy group; an aryloxy group; an alkylthioxy group; an arylthioxy group; an alkylsulfoxy group; an arylsulfoxy group; an alkenyl group; a silyl group; a siloxane group; a boron group; an amine group; an arylphosphine group; a phosphine oxide group; an aryl group; and a heterocyclic group, or being substituted with a substituent to which two or more substituents among the exemplified substituents are linked, or having no substituent. For example, “the substituent to which two or more substituents are linked” may be a biphenyl group. That is, the biphenyl group may also be an aryl group, and may be interpreted as a substituent to which two phenyl groups are linked.

In the present specification, a halogen group may be fluorine, chlorine, bromine or iodine.

In the present specification, the number of carbon atoms of an imide group is not particularly limited, but is preferably 1 to 30.

In the present specification, for an amide group, the nitrogen of the amide group may be substituted with hydrogen, a straight-chained, branched, or cyclic alkyl group having 1 to 30 carbon atoms, or an aryl group having 6 to 30 carbon atoms.

In the present specification, for a carbonyl group, the carbon atom of a carbonyl group may be substituted with a straight-chained, branched, or cyclic alkyl group preferably having 1 to 30 carbon atoms, or an aryl group preferably having 6 to 30 carbon atoms.

In the present specification, for an ester group, the oxygen of the ester group may be substituted with a straight-chained, branched, or cyclic alkyl group having 1 to 25 carbon atoms, or an aryl group having 6 to 30 carbon atoms.

In the present specification, the alkyl group may be straight-chained or branched, and the number of carbon atoms thereof is not particularly limited, but is preferably 1 to 30. Specific examples thereof include methyl, ethyl, propyl, n-propyl, isopropyl, butyl, n-butyl, isobutyl, tert-butyl, sec-butyl, 1-methyl-butyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present specification, a cycloalkyl group is not particularly limited, but has preferably 3 to 30 carbon atoms, and specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present specification, the alkoxy group may be straight-chained, branched, or cyclic. The number of carbon atoms of the alkoxy group is not particularly limited, but is preferably 1 to 30. Specific examples thereof include methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, an amine group may be selected from the group consisting of —NH₂; an alkylamine group; an N-arylalkylamine group; an arylamine group; an N-arylheteroarylamine group; an N-alkylheteroarylamine group; and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, an N-phenylnaphthylamine group, a ditolylamine group, an N-phenyltolylamine group, a triphenylamine group, and the like, but are not limited thereto.

In the present specification, an N-alkylarylamine group means an amine group in which a N of the amine group are substituted with an alkyl group and an aryl group.

In the present specification, an N-arylheteroarylamine group means an amine group in which a N of the amine group are substituted with an aryl group and a heteroaryl group.

In the present specification, an N-alkylheteroarylamine group means an amine group in which a N of the amine group are substituted with alkyl group and a heteroaryl group.

In the present specification, the alkyl group in the alkylamine group, the N-arylalkylamine group, the alkylthioxy group, the alkylsulfoxy group, and the N-alkylheteroarylamine group is the same as the above-described examples of the alkyl group. Specifically, examples of the alkylthioxy group include a methylthioxy group, an ethylthioxy group, a tert-butylthioxy group, a hexylthioxy group, an octylthioxy group, and the like, and examples of the alkylsulfoxy group include methylsulfoxy group, an ethylsulfoxy group, a propylsulfoxy group, a butylsulfoxy group, and the like, but the examples are not limited thereto.

In the present specification, the alkenyl group may be straight-chained or branched, and the number of carbon atoms thereof is not particularly limited, but is preferably 2 to 30. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, specific examples of a silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be —BR₁₀₀R₂₀₀, and R₁₀₀ and R₂₀₀ are the same as or different from each other, and may be each independently selected from the group consisting of hydrogen; deuterium; halogen; a nitrile group; a substituted or unsubstituted monocyclic or polycyclic cycloalkyl group having 3 to 30 carbon atoms; a substituted or unsubstituted straight-chained or branched alkyl group having 1 to 30 carbon atoms; a substituted or unsubstituted monocyclic or polycyclic aryl group having 6 to 30 carbon atoms; and a substituted or unsubstituted monocyclic or polycyclic heteroaryl group having 2 to 30 carbon atoms.

In the present specification, specific examples of a phosphine oxide group include a diphenylphosphine oxide group, dinaphthylphosphine oxide group, and the like, but are not limited thereto.

In the present specification, an aryl group may be monocyclic or polycyclic.

When the aryl group is a monocyclic aryl group, the number of carbon atoms thereof is not particularly limited, but is preferably 6 to 30. Specific examples of the monocyclic aryl group include a phenyl group, a biphenyl group, a terphenyl group, and the like, but are not limited thereto.

When the aryl group is a polycyclic aryl group, the number of carbon atoms thereof is not particularly limited, but is preferably 10 to 30. Specific examples of the polycyclic aryl group include a naphthyl group, an anthracenyl group, a phenanthryl group, a pyrenyl group, a perylenyl group, a chrysenyl group, a fluorenyl group, and the like, but are not limited thereto.

In the present specification, the fluorenyl group may be substituted, and adjacent substituents may be bonded to each other to form a ring.

When the fluorenyl group is substituted, the fluorenyl group may be

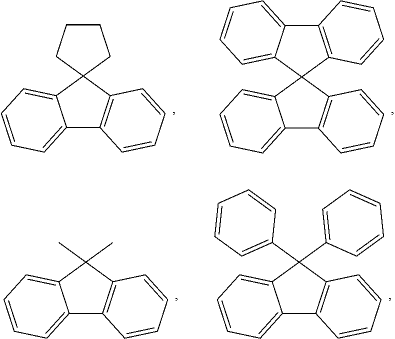

and the like. However, the fluorenyl group is not limited thereto.

In the present specification, the aryl group in the aryloxy group, the arylthioxy group, the arylsulfoxy group, the N-arylalkylamine group, the N-arylheteroarylamine group, and the arylphosphine group is the same as the above-described examples of the aryl group. Specifically, examples of the aryloxy group include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group, and the like, examples of the arylthioxy group include a phenylthioxy group, a 2-methylphenylthioxy group, a 4-tert-butylphenylthioxy group, and the like, and examples of the arylsulfoxy group include a benzenesulfoxy group, a p-toluenesulfoxy group, and the like, but the examples are not limited thereto.

In the present specification, examples of the arylamine group include a substituted or unsubstituted monoarylamine group, a substituted or unsubstituted diarylamine group, or a substituted or unsubstituted triarylamine group. The aryl group in the arylamine group may be a monocyclic aryl group or a polycyclic aryl group. The arylamine group including two or more aryl groups may include a monocyclic aryl group, a polycyclic aryl group, or both a monocyclic aryl group and a polycyclic aryl group. For example, the aryl group in the arylamine group may be selected from the above-described examples of the aryl group.

In the present specification, a heterocyclic group includes one or more atoms other than carbon, that is, one or more heteroatoms, and specifically, the heteroatom may include one or more atoms selected from the group consisting of O, N, Se, S, and the like. The number of carbon atoms thereof is not particularly limited, but is preferably 2 to 30, and the heterocyclic group may be monocyclic or polycyclic. Examples of the heterocyclic group include a thiophene group, a furanyl group, a pyrrole group, an imidazolyl group, a thiazolyl group, an oxazolyl group, an oxadiazolyl group, a pyridyl group, a bipyridyl group, a pyrimidyl group, a triazinyl group, a triazolyl group, an acridyl group, a pyridazinyl group, a pyrazinyl group, a quinolinyl group, a quinazolinyl group, a quinoxalinyl group, a phthalazinyl group, a pyridopyrimidyl group, a pyridopyrazinyl group, a pyrazinopyrazinyl group, an isoquinolinyl group, an indolyl group, a carbazolyl group, a benzoxazolyl group, a benzimidazolyl group, a benzothiazolyl group, a benzocarbazolyl group, a benzothiophene group, a dibenzothiophene group, a benzofuranyl group, a phenanthrolinyl group (phenanthroline), a triazolyl group, an isoxazolyl group, an oxadiazolyl group, a thiadiazolyl group, a phenothiazinyl group, a dibenzofuranyl group, and the like, but are not limited thereto.

In the present specification, examples of the heteroarylamine group include a substituted or unsubstituted monoheteroarylamine group, a substituted or unsubstituted diheteroarylamine group, or a substituted or unsubstituted triheteroarylamine group. The heteroarylamine group including two or more heteroaryl groups may include a monocyclic heteroaryl group, a polycyclic heteroaryl group, or both a monocyclic heteroaryl group and a polycyclic heteroaryl group. For example, the heteroaryl group in the heteroarylamine group may be selected from the above-described examples of the heterocyclic group.

In the present specification, examples of the heteroaryl group in the N-arylheteroarylamine group and the N-alkylheteroarylamine group are the same as the above-described examples of the heterocyclic group.

In an exemplary embodiment of the present specification, X1 to X3 are the same as or different from each other, and are each independently S, O, Se, Te, NR, CRR′, SiRR′, PR, or GeRR′, and R and R′ are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heterocyclic group.

In an exemplary embodiment of the present specification, X1 to X3 are the same as or different from each other, and are each independently S, NR, or CRR′, and R and R′ are the same as those described above.

In an exemplary embodiment of the present specification, X1 to X3 are each S.

Since the molecule exhibits planarity due to the fixed conformation lock characteristics between 0 and Y1 (and/or Y2) and between R11 (and/or R12) and Y3 (and/or Y4) in a benzo[1,2-c:4,5-c′]dithiophene-4,8-dione group, the compound according to an exemplary embodiment of the present specification exhibits strong aggregation characteristics, and has improved crystallinity. Further, the pi-pi interaction in the compounds is so strong that the charge transfer caused by hopping is increased.

In addition, the compound according to an exemplary embodiment of the present specification may absorb light at various wavelengths by including both a benzo[1,2-c:4,5-c′]dithiophene-4,8-dione group having a weak electron-withdrawing property and a benzothiadiazole group having a strong electron-withdrawing property. That is, the compound may exhibit an effect of increasing an amount of light absorbed.

In an exemplary embodiment of the present specification, p and q are the same as each other, and are each independently an integer from 0 to 3, and when p and q are each 2 or more, the structures in the parenthesis are the same as or different from each other.

In an exemplary embodiment of the present specification, p and q are the same as each other, and are each 0 or 1.

In an exemplary embodiment of the present specification, p and q are 0.

In an exemplary embodiment of the present specification, p and q are 1.

In an exemplary embodiment of the present specification, r and s are the same as or different from each other, and are each independently an integer from 1 to 3, and when r and s are each 2 or more, the structures in the parenthesis are the same as or different from each other.

In an exemplary embodiment of the present specification, r and s are the same as each other, and are each 1 or 2.

In an exemplary embodiment of the present specification, r and s are 1.

In an exemplary embodiment of the present specification, r and s are 2.

In an exemplary embodiment of the present specification, Formula 1 is represented by any one of the following Formulae 1-1 to 1-4.

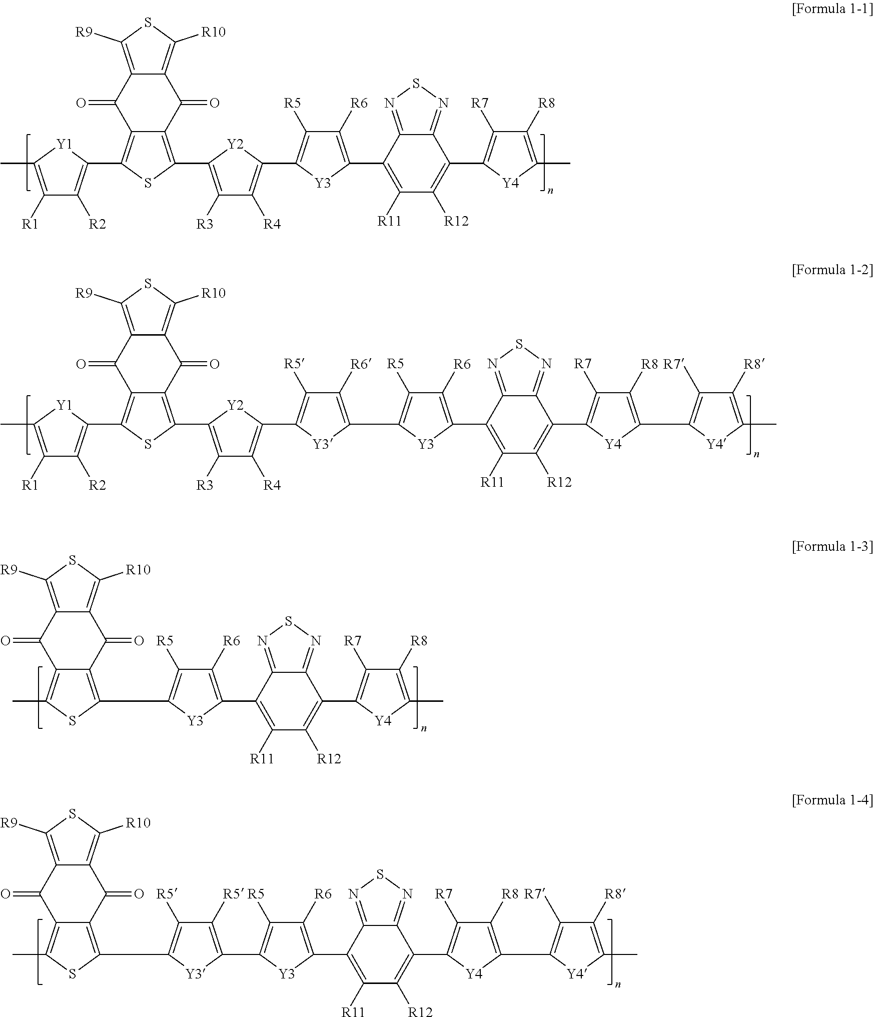

In Formulae 1-1 to 1-4,

n, R1 to R12, and Y1 to Y4 are the same as those defined in Formula 1,

Y3′ and Y4′ are the same as or different from each other, and are each independently S, O, Se, Te, NR, CRR′, SiRR′, PR, or GeRR′, and

R5′, R6′, R7′, R8′, R, and R′ are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heterocyclic group.

In an exemplary embodiment of the present specification, R9 and R10 are the same as or different from each other, and are each independently a substituted or unsubstituted alkyl group; a substituted or unsubstituted cycloalkyl group; or a substituted or unsubstituted aryl group.

In an exemplary embodiment of the present specification, R9 and R10 are the same as or different from each other, and are each independently a substituted or unsubstituted alkyl group.

In an exemplary embodiment of the present specification, R9 and R10 are the same as or different from each other, and are each independently a straight-chained or branched alkyl group.

In an exemplary embodiment of the present specification, R9 and R10 are the same as or different from each other, and are each independently a branched alkyl group having 1 to 30 carbon atoms.

In an exemplary embodiment of the present specification, R9 and R10 are the same as or different from each other, and are each independently a branched alkyl group having 1 to 15 carbon atoms.

In an exemplary embodiment of the present specification, R9 and R10 are a 2-ethylhexyl group.

In an exemplary embodiment of the present specification, Formula 1 is represented by the following Formula 2.

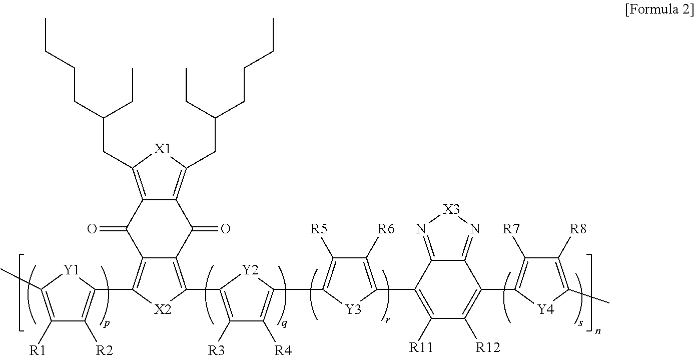

In Formula 2, p, q, r, s, n, R1 to R8, R11, R12, X1 to X3, and Y1 to Y4 are the same as those defined in Formula 1.

In an exemplary embodiment of the present specification, Y1 to Y4 are the same as or different from each other, and are each independently S, O, Se, Te, NR, CRR′, SiRR′, PR, or GeRR′, and R and R′ are the same as those described above.

In an exemplary embodiment of the present specification, Y1 to Y4 are the same as or different from each other, and are each independently S, NR, or CRR′, and R and R′ are the same as those described above.

In an exemplary embodiment of the present specification, Y1 to Y4 are each S.

In an exemplary embodiment of the present specification, R1 to R8 are the same as or different from each other, and are each independently hydrogen; a halogen group; a substituted or unsubstituted alkyl group; a substituted or unsubstituted alkoxy group; a substituted or unsubstituted aryl group; or a substituted or unsubstituted heterocyclic group.

In an exemplary embodiment of the present specification, R1 to R8 are the same as or different from each other, and are each independently hydrogen; a halogen group; a substituted or unsubstituted alkyl group; or a substituted or unsubstituted alkoxy group.

In an exemplary embodiment of the present specification, R2, R3, and R5 to R8 are hydrogen.

In an exemplary embodiment of the present specification, Formula 1 is any one of the following Formulae 2-1 to 2-4.

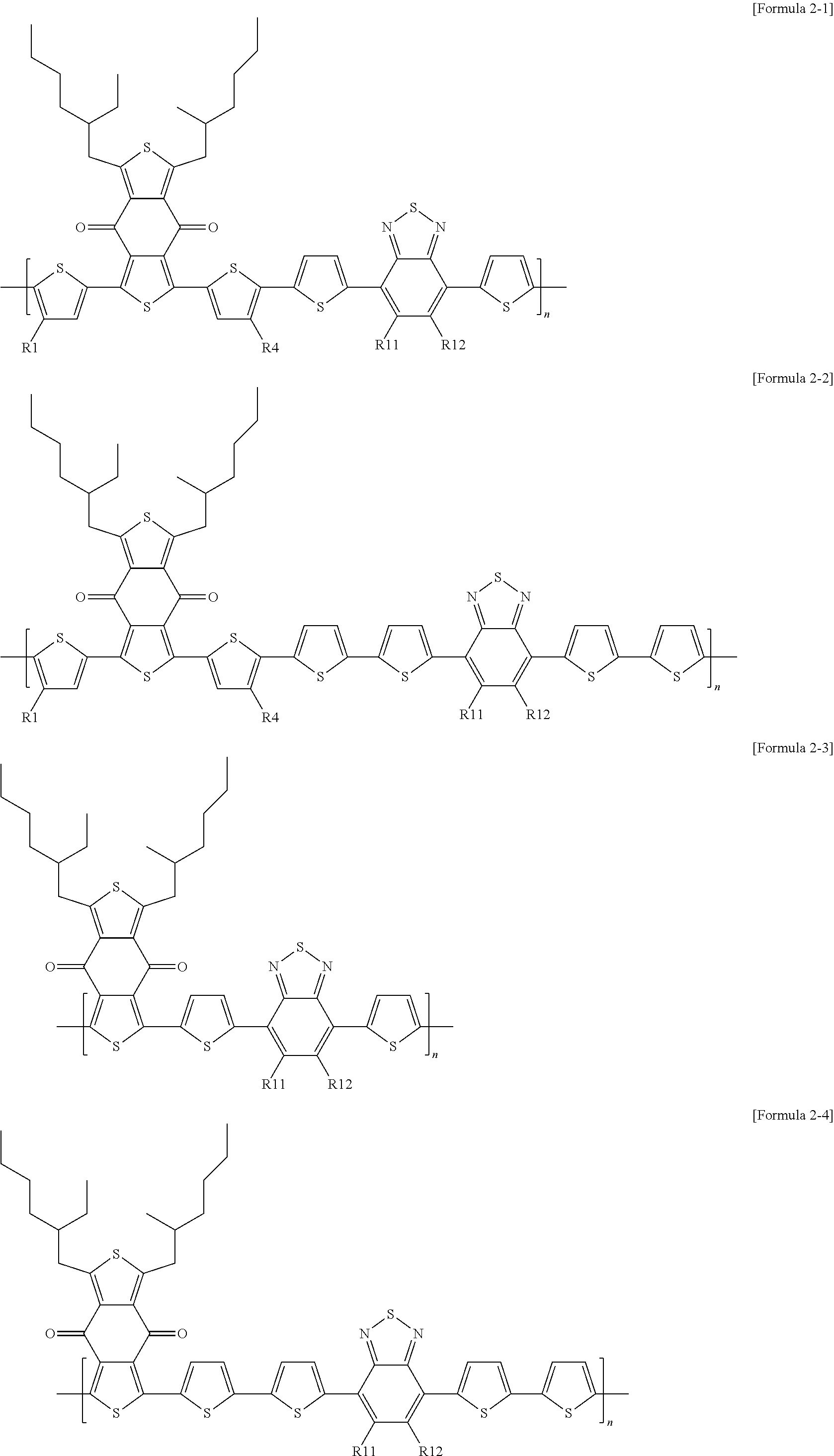

In Formulae 2-1 to 2-4,

R1, R4, R11, R12, and n are the same as those defined in Formula 1.

In an exemplary embodiment of the present specification, R1 and R4 are the same as or different from each other, and are each independently hydrogen or a substituted or unsubstituted alkyl group.

In an exemplary embodiment of the present specification, R1 and R4 are the same as or different from each other, and are each independently hydrogen; or a straight-chained or branched alkyl group.

In an exemplary embodiment of the present specification, R1 and R4 are the same as or different from each other, and are each independently hydrogen; or a branched alkyl group having 1 to 30 carbon atoms.

In an exemplary embodiment of the present specification, R1 and R4 are each hydrogen.

In an exemplary embodiment of the present specification, R1 and R4 are each a 2-octyldodecane group.

In an exemplary embodiment of the present specification, R11 and R12 are the same as or different from each other, and are each independently hydrogen; a halogen group; a substituted or unsubstituted alkyl group; a substituted or unsubstituted alkoxy group; a substituted or unsubstituted aryl group; or a substituted or unsubstituted heterocyclic group.

In an exemplary embodiment of the present specification, R11 and R12 are the same as or different from each other, and are each independently hydrogen; a halogen group; or a substituted or unsubstituted alkoxy group.

In an exemplary embodiment of the present specification, R11 and R12 are the same as or different from each other, and are each independently a halogen group.

In an exemplary embodiment of the present specification, R11 and R12 are each fluorine.

In an exemplary embodiment of the present specification, R11 is fluorine, and R12 is hydrogen.

In an exemplary embodiment of the present specification, R11 and R12 are the same as or different from each other, and are each independently a substituted or unsubstituted alkoxy group.

In an exemplary embodiment of the present specification, R11 and R12 are the same as or different from each other, and are each independently an alkoxy group having 1 to 30 carbon atoms.

In an exemplary embodiment of the present specification, R11 and R12 are the same as or different from each other, and are each independently an alkoxy group having 1 to 15 carbon atoms.

In an exemplary embodiment of the present specification, Formula 1 is represented by any one of the following compounds.

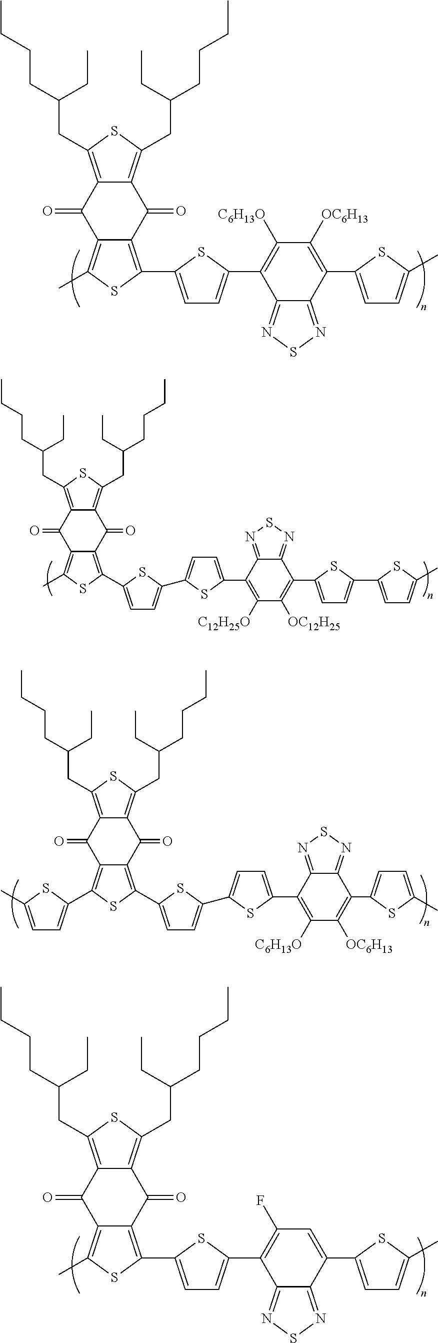

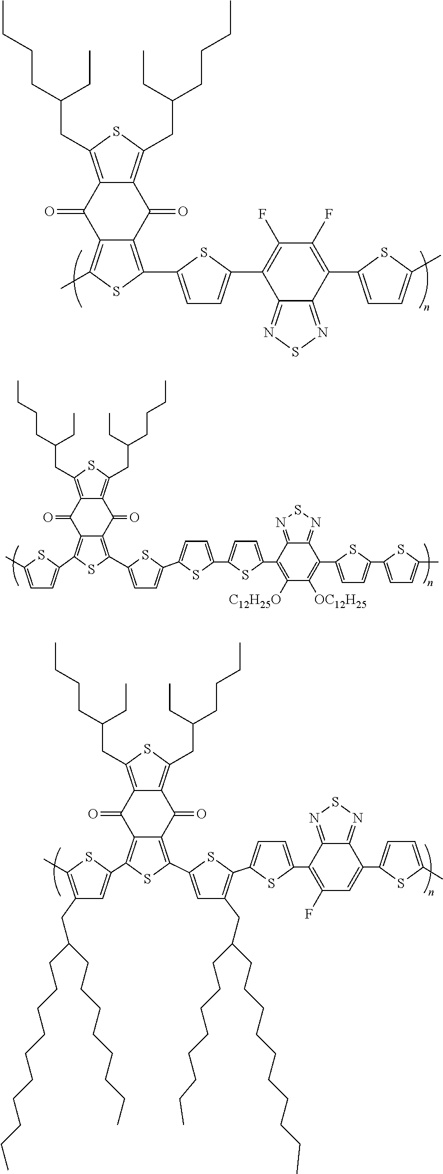

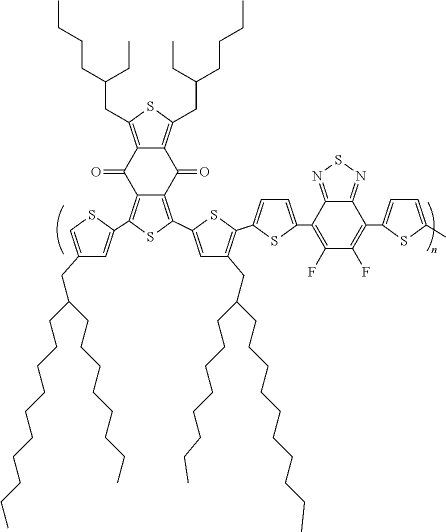

In the compounds,

n is an integer from 1 to 10,000.

In an exemplary embodiment of the present specification, an end group of the compound is a substituted or unsubstituted aryl group.

In an exemplary embodiment of the present specification, an end group of the compound is benzotrifluoride.

An exemplary embodiment of the present specification provides an organic solar cell including: a first electrode;

a second electrode provided to face the first electrode; and

an organic material layer having one or more layers provided between the first electrode and the second electrode and including a photoactive layer,

in which one or more layers of the organic material layer include the compound.

In an exemplary embodiment of the present specification, the organic solar cell may further include an additional organic material layer. The organic solar cell may reduce the number of organic material layers by using an organic material which simultaneously has various functions.

In an exemplary embodiment of the present specification, the organic solar cell includes a first electrode, a photoactive layer, and a second electrode. The organic solar cell may further include a substrate, a hole transport layer, and/or an electron transport layer.

FIG. 1 illustrates an organic solar cell according to an exemplary embodiment of the present specification. Specifically, FIG. 1 illustrates an organic solar cell in which a substrate, a first electrode, a hole transport layer, a photoactive layer, and a second electrode are sequentially laminated.

In an exemplary embodiment of the present specification, the photoactive layer includes the compound.

In an exemplary embodiment of the present specification, the organic material layer includes a hole transport layer, a hole injection layer, or a layer which simultaneously transports and injects holes, and the hole transport layer, the hole injection layer, or the layer which simultaneously transports and injects holes includes the compound.

In another exemplary embodiment, the organic material layer includes an electron injection layer, an electron transport layer, or a layer which simultaneously injects and transports electrons, and the electron injection layer, the electron transport layer, or the layer which simultaneously injects and transports electrons includes the compound.

In an exemplary embodiment of the present specification, the first electrode is an anode, and the second electrode is a cathode. In another exemplary embodiment of the present specification, the first electrode is a cathode, and the second electrode is an anode.

In an exemplary embodiment of the present specification, in the organic solar cell, a cathode, a photoactive layer, and an anode may be arranged in this order, and an anode, a photoactive layer, and a cathode may be arranged in this order, but the arrangement order is not limited thereto.

In another exemplary embodiment, in the organic solar cell, an anode, a hole transport layer, a photoactive layer, an electron transport layer, and a cathode may also be arranged in this order, and a cathode, an electron transport layer, a photoactive layer, a hole transport layer, and an anode may also be arranged in this order, but the arrangement order is not limited thereto.

In an exemplary embodiment of the present specification, the photoactive layer includes an electron donor and an electron acceptor, and the electron donor includes the compound.

In an exemplary embodiment of the present specification, a material for the electron acceptor may be selected from the group consisting of fullerene, fullerene derivatives, bathocuproine, semi-conducting elements, semi-conducting compounds, and combinations thereof. Specifically, the material for the electron acceptor may be phenyl C₆₀-butyric acid methyl ester (PC₆₀BM), phenyl C₆₁-butyric acid methyl ester (PC₆₁BM), or phenyl C₇₁-butyric acid methyl ester (PC₇₁BM).

In an exemplary embodiment of the present specification, the electron donor and the electron acceptor constitute a bulk heterojunction (BHJ). A material for the electron donor and a material for the electron acceptor may be mixed at a ratio (w/w) of 1:10 to 10:1. Specifically, the material for the electron donor and the material for the electron acceptor may be mixed at a ratio (w/w) of 1:1 to 1:10, and more specifically, the material for the electron donor and the material for the electron acceptor may be mixed at a ratio (w/w) of 1:1 to 1:5. If necessary, the material for the electron donor and the material for the electron acceptor may be mixed at a ratio (w/w) of 1:1 to 1:3.

In an exemplary embodiment of the present specification, the photoactive layer has a bilayer thin film structure including an n-type organic material layer and a p-type organic material layer, and the p-type organic material layer includes the compound.

In the present specification, the substrate may be a glass substrate or a transparent plastic substrate having excellent transparency, surface smoothness, ease of handling, and waterproofing properties, but is not limited thereto, and the substrate is not limited as long as the substrate is typically used in the organic solar cell. Specific examples thereof include glass or polyethylene terephthalate (PET), polyethylene naphthalate (PEN), polypropylene (PP), polyimide (PI), triacetyl cellulose (TAC), and the like, but are not limited thereto.

The first electrode may be a material which is transparent and has excellent conductivity, but is not limited thereto. Examples thereof include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer, such as poly(3-methylthiophene), poly[3,4-(ethylene-1,2-dioxy)thiophene] (PEDOT), polypyrrole, and polyaniline; and the like, but are not limited thereto.

A method of forming the first electrode is not particularly limited, but the first electrode may be formed, for example, by being applied onto one surface of a substrate using a method such as sputtering, e-beam, thermal deposition, spin coating, screen printing, inkjet printing, doctor blade, or gravure printing, or by being coated in the form of a film.

When the first electrode is formed on a substrate, the first electrode may be subjected to processes of cleaning, removing moisture, and hydrophilic modification.

For example, a patterned ITO substrate is sequentially cleaned with a cleaning agent, acetone, and isopropyl alcohol (IPA), and then dried on a hot plate at 100° C. to 150° C. for 1 to 30 minutes, preferably at 120° C. for 10 minutes in order to remove moisture, and when the substrate is completely cleaned, the surface of the substrate is hydrophilically modified.

Through the surface modification as described above, the junction surface potential may be maintained at a level suitable for a surface potential of a photoactive layer. Further, during the modification, a polymer thin film may be easily formed on the first electrode, and the quality of the thin film may also be improved.

Examples of a pre-treatment technology for the first electrode include a) a surface oxidation method using a parallel flat plate-type discharge, b) a method of oxidizing the surface through ozone produced by using UV rays in a vacuum state, c) an oxidation method using oxygen radicals produced by plasma, and the like.

One of the methods may be selected according to the state of the first electrode or the substrate. However, although any method is used, it is preferred to commonly prevent oxygen from being separated from the surface of the first electrode or the substrate, and maximally inhibit moisture and organic materials from remaining. In this case, it is possible to maximize a substantial effect of the pre-treatment.

As a specific example, it is possible to use a method of oxidizing the surface through ozone produced by using UV. In this case, a patterned ITO substrate after being ultrasonically cleaned is baked on a hot plate and dried well, and then introduced into a chamber, and the patterned ITO substrate may be cleaned by ozone generated by allowing an oxygen gas to react with UV light by operating a UV lamp.

However, the surface modification method of the patterned ITO substrate in the present specification need not be particularly limited, and any method may be used as long as the method is a method of oxidizing a substrate.

The second electrode may be a metal having a low work function, but is not limited thereto. Specific examples thereof include: a metal such as magnesium, calcium, sodium, potassium, titanium, indium, yttrium, lithium, gadolinium, aluminum, silver, tin, and lead, or an alloy thereof; and a multi-layer structured material, such as LiF/Al, LiO₂/Al, LiF/Fe, Al:Li, Al:BaF₂, and Al:BaF₂:Ba, but are not limited thereto.

The second electrode may be deposited and formed in a thermal evaporator showing a vacuum degree of 5×10⁻⁷ torr or less, but the forming method is not limited to this method.

A material for the hole transport layer and/or a material for the electron transport layer serve to efficiently transfer electrons and holes separated from a photoactive layer to an electrode, and the materials are not particularly limited.

The material for the hole transport layer may be poly(3,4-ethylenedioxythiophene) doped with poly(styrenesulfonic acid) (PEDOT:PSS) and molybdenum oxide (MoOₓ); vanadium oxide (V₂O₅); nickel oxide (NiO); tungsten oxide (WOₓ); and the like, but is not limited thereto.

The material for the electron transport layer may be electron-extracting metal oxides, and specific examples thereof include: metal complexes of 8-hydroxyquinoline; complexes including Alq₃; metal complexes including Liq; LiF; Ca; titanium oxide (TiOₓ); zinc oxide (ZnO); cesium carbonate (Cs₂CO₃); and the like, but are not limited thereto.

The photoactive layer may be formed by dissolving a photoactive material such as an electron donor and/or an electron acceptor in an organic solvent, and then applying the solution by a method such as spin coating, dip coating, screen printing, spray coating, doctor blade, and brush painting, but the forming method is not limited thereto.

### MODE FOR INVENTION

A preparation method of the compound and the manufacture of an organic solar cell including the same will be described in detail in the following Preparation Examples and Examples. However, the following Examples are provided for exemplifying the present specification, and the scope of the present specification is not limited thereby.

### Preparation Example 1. Preparation of Compound 1

(1) Preparation of Compound C

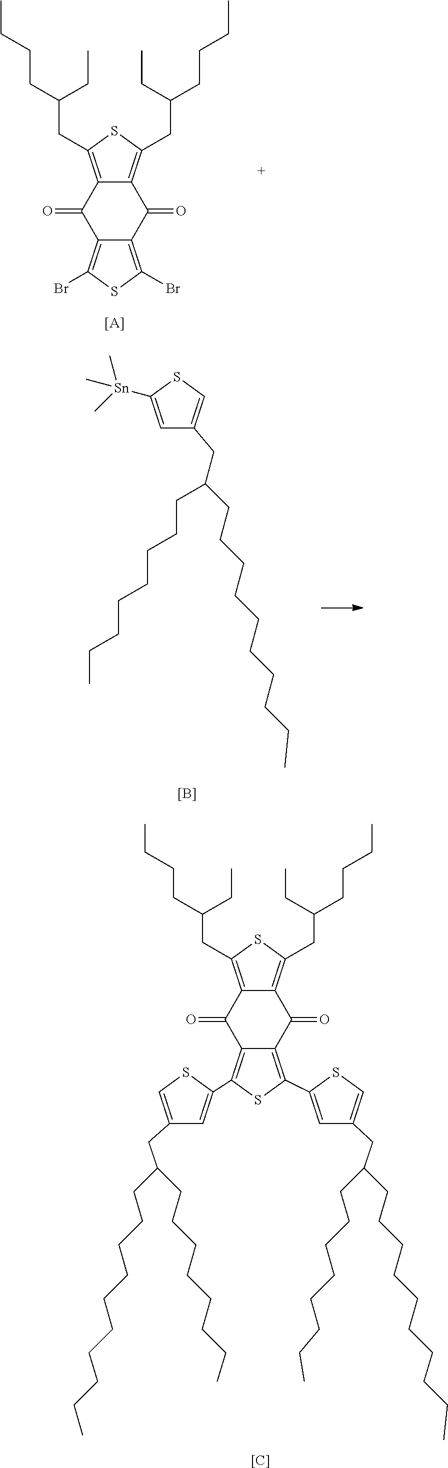

Compound A (1.41 g, 2.34 mmol), Compound B (3.08 g, 5.85 mmol), and tetrakis(triphenylphosphine)palladium(0)(Pd(PPh₃)₄) (0.127 g, 0.11 mmol) were put into a solution in which 100 mL of toluene and 100 mL of dimethylformamide (DMF) were mixed, and the resulting mixture was allowed to react at 110° C. for 48 hours. After the reaction, the solution was cooled, extraction was performed with dichloromethane (DCM), and then the solvent was removed. Thereafter, the product was purified with flash chromatography (hexane:methyl chloride=5:1), thereby obtaining Compound C (yield: 56%).

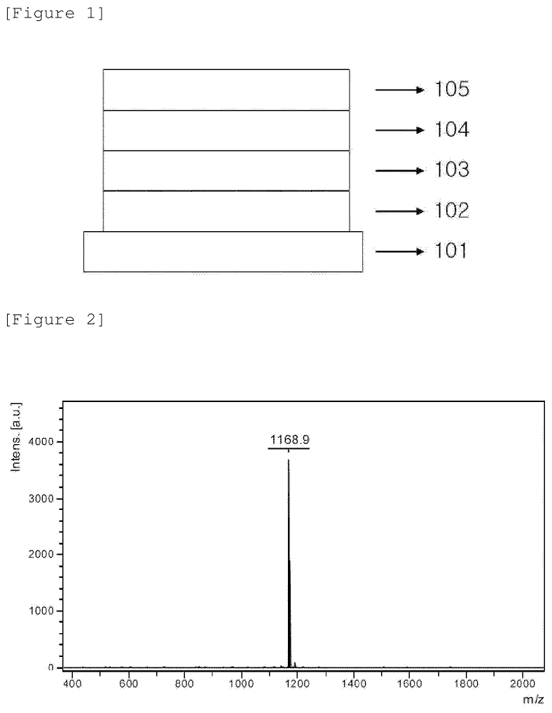

FIG. 2 is a view illustrating an MS measurement result of Compound C.

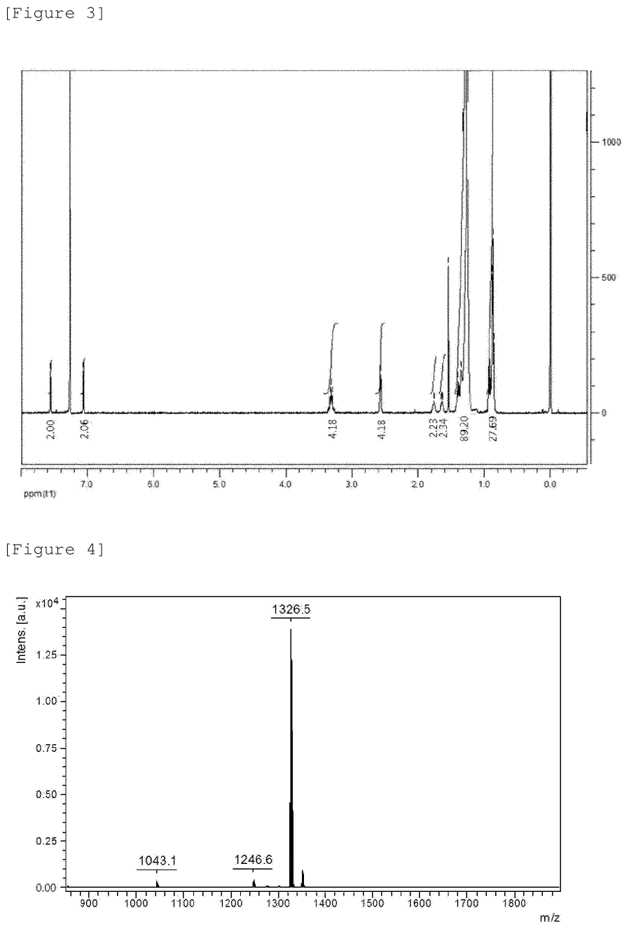

FIG. 3 is a view illustrating an NMR measurement result of Compound C.

(2) Preparation of Compound D

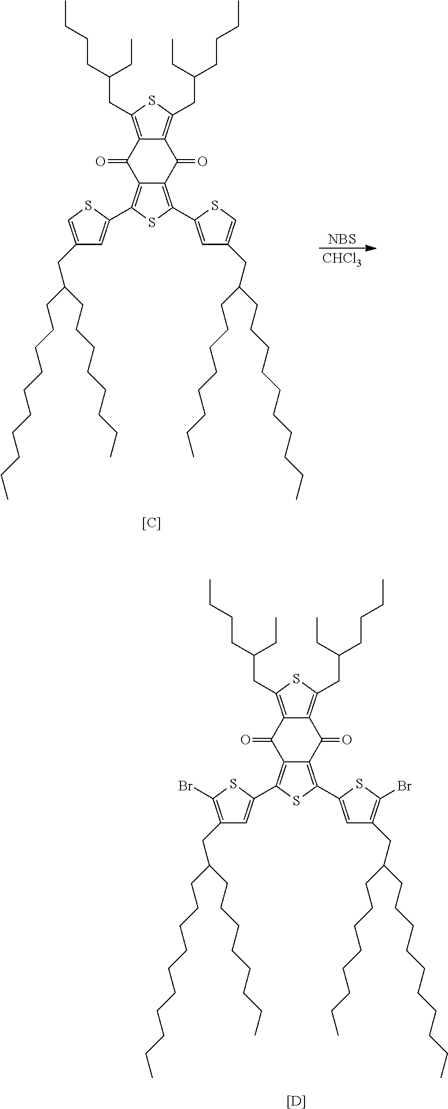

After Compound C (1.53 g, 1.31 mmol) and N-bromosuccinimide (NBS) (0.50 g, 2.82 mmol) were put into 5 mL of chloroform (CHCl₃) at 0° C., the resulting mixture was stirred at room temperature for 24 hours. After the reaction, the solution was cooled, extraction was performed with dichloromethane (DCM), and then the solvent was removed. Thereafter, the product was purified with flash chromatography using hexane, recrystallized through isopropyl alcohol (IPA), and filtered. The produced solid was washed with IPA and methanol, and then dried under a vacuum condition for 24 hours, thereby obtaining Compound D (yield: 83%).

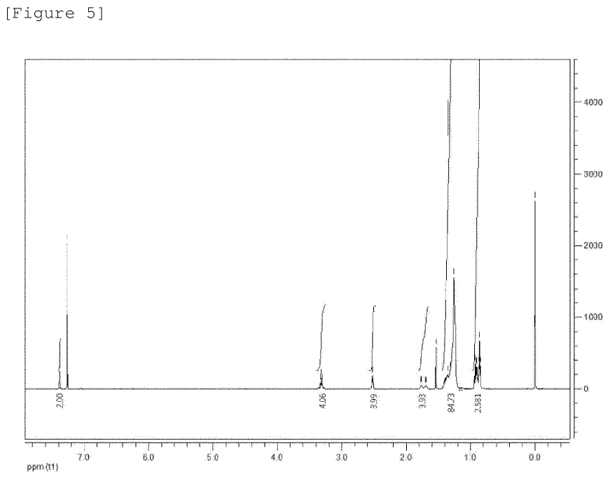

FIG. 4 is a view illustrating an MS measurement result of Compound D.

FIG. 5 is a view illustrating an NMR measurement result of Compound D.

(3) Preparation of Compound 1

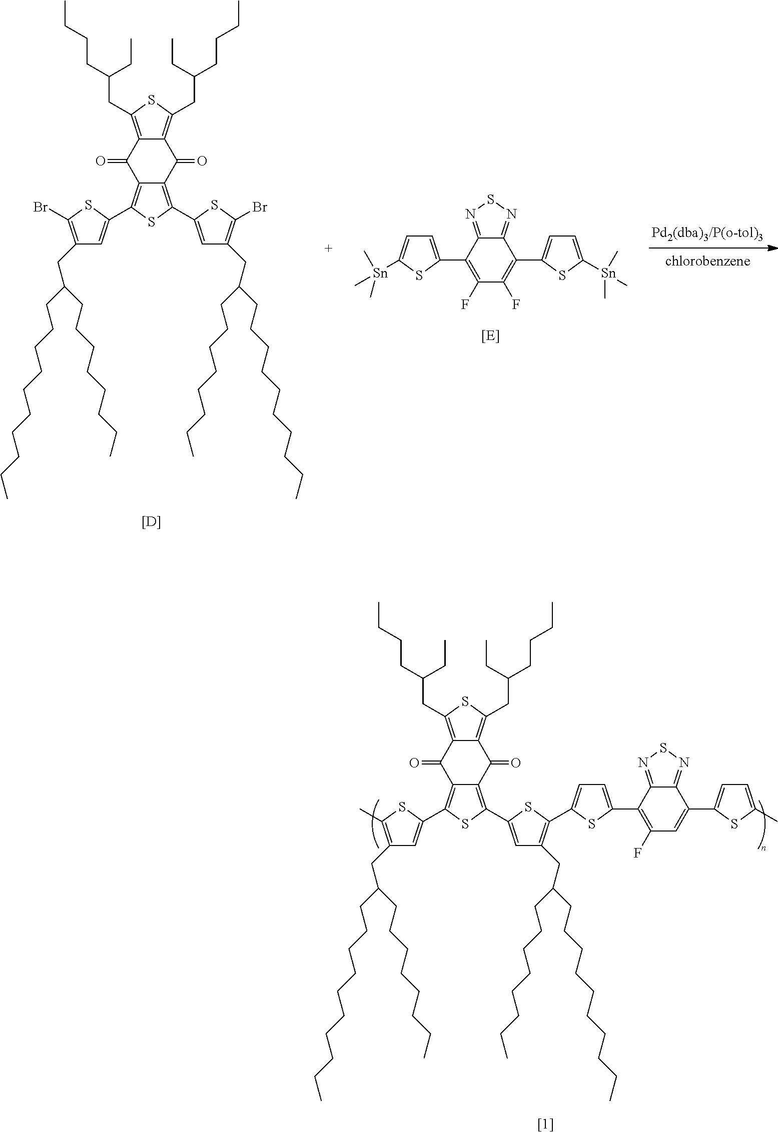

Compound D (0.53 g, 0.4 mmol), Compound E (0.265 g, 0.4 mmol), and 10 mL of chlorobenzene (CB) were put into a 100-mL flask under a nitrogen (N₂) atmosphere, the mixture was bubbled with nitrogen for 30 minutes, and then tris(dibenzylideneacetone)dipalladium(0) (Pd₂(dba)₃) (7.3 mg, 0.008 mmol) and tri(o-tolyl)phosphine (P(o-tol)₃) (9.7 mg, 0.032 mmol) were put thereinto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 1 (yield: 80%).

### Preparation Example 2. Preparation of Compound 2

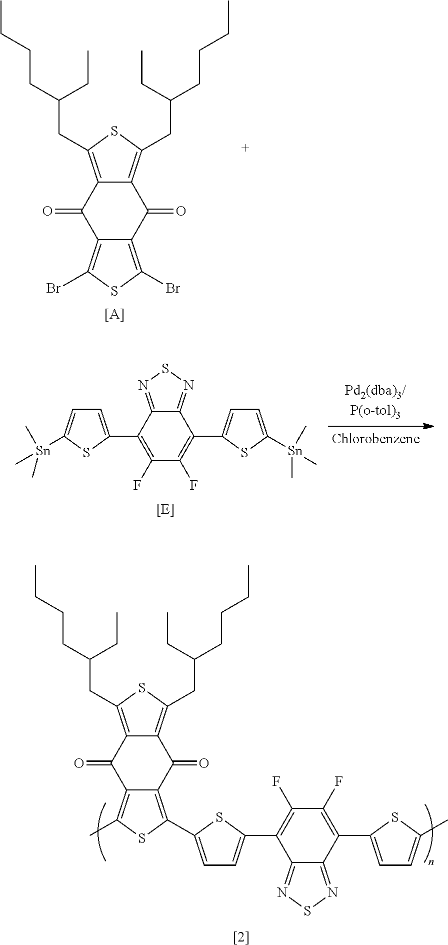

Compound A (0.24 g, 0.4 mmol), Compound E (0.265 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were added thereto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 2 (yield: 84.5%).

### Preparation Example 3. Preparation of Compound 3

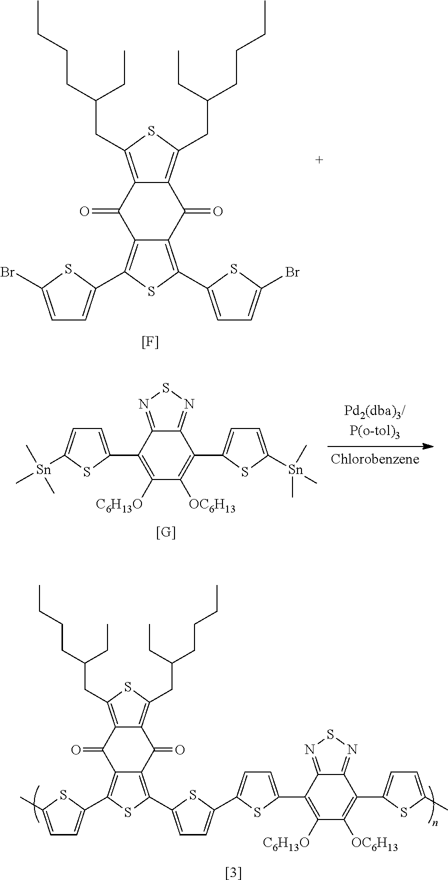

Compound F (0.306 g, 0.4 mmol), Compound G (0.33 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were put thereinto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 3 (yield: 57%).

### Preparation Example 4. Preparation of Compound 4

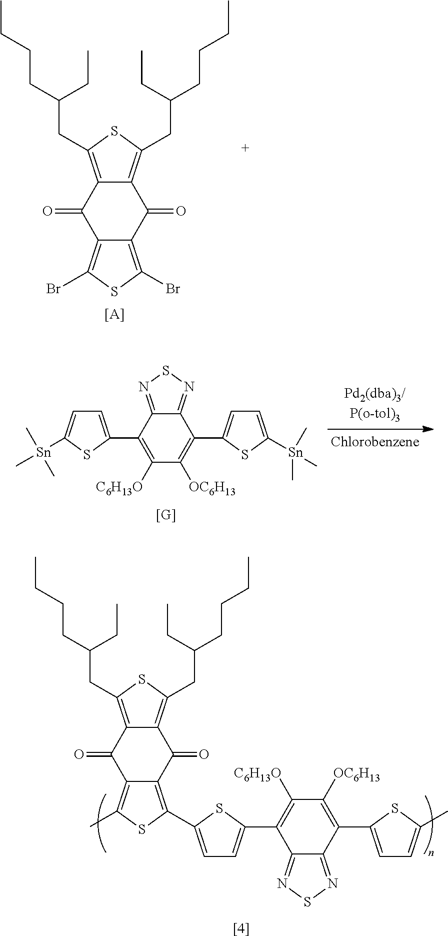

Compound A (0.24 g, 0.4 mmol), Compound G (0.33 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were put thereinto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 4 (yield: 71%).

### Preparation Example 5. Preparation of Compound 5

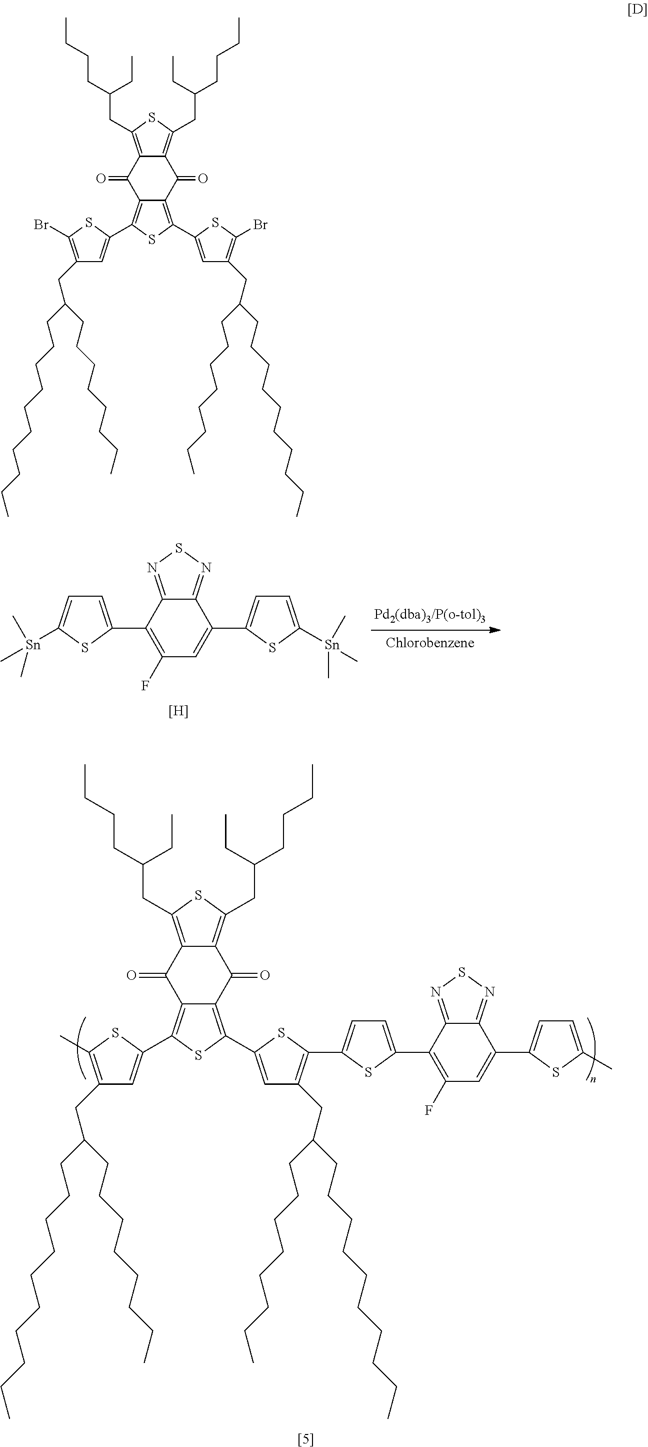

Compound D (0.53 g, 0.4 mmol), Compound H (0.258 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were put thereinto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 5 (yield: 48%).

### Preparation Example 6. Preparation of Compound 6

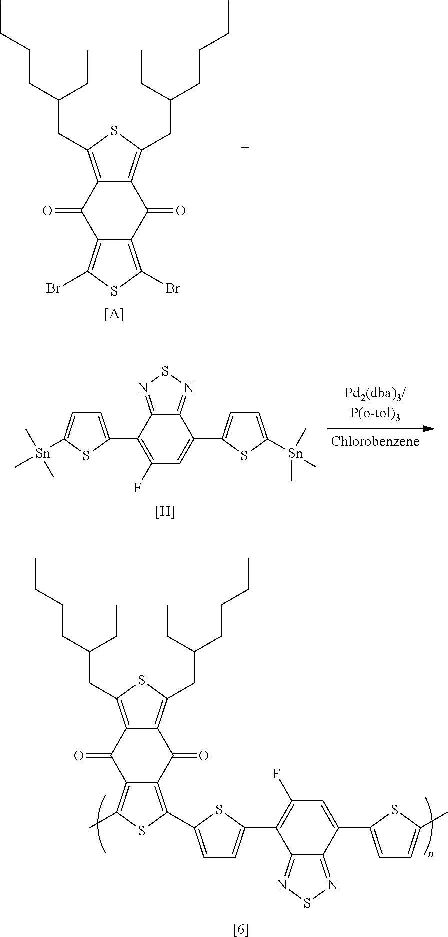

Compound A (0.24 g, 0.4 mmol), Compound H (0.258 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were put thereinto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 6 (yield: 78%).

### Preparation Example 7. Preparation of Compound 7

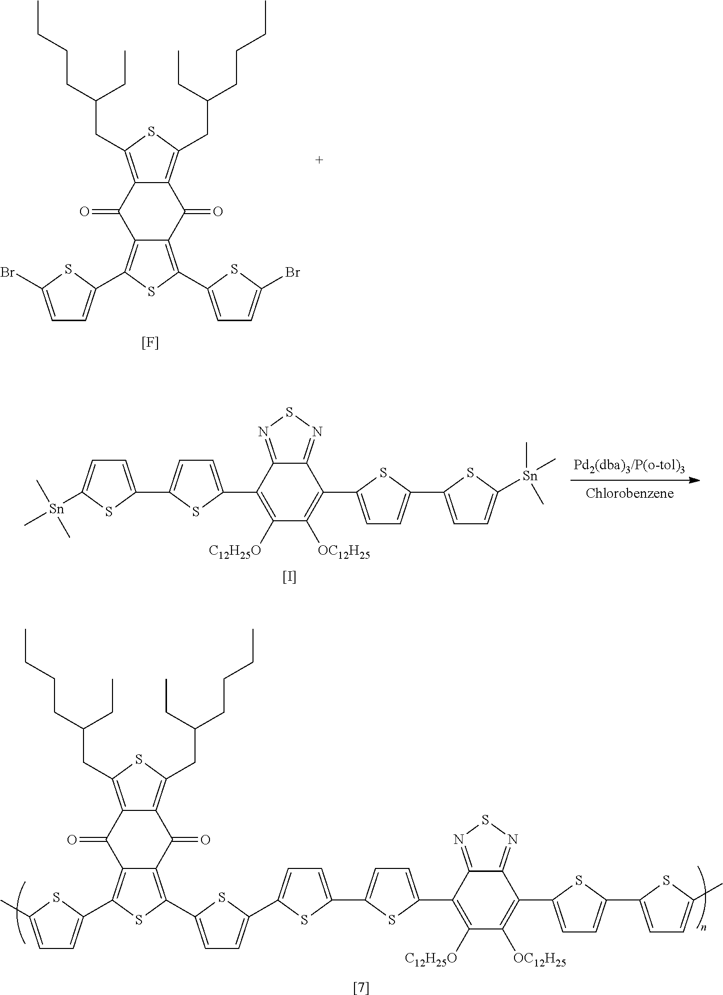

Compound F (0.306 g, 0.4 mmol), Compound I (0.46 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were put thereinto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 7 (yield: 57%).

### Preparation Example 8. Preparation of Compound 8

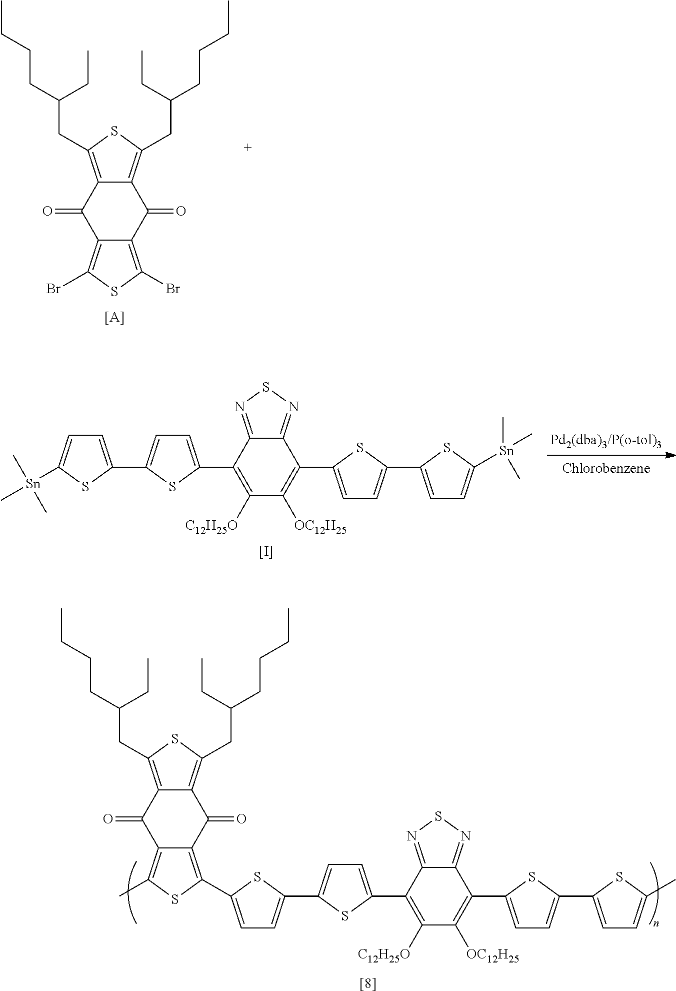

Compound A (0.24 g, 0.4 mmol), Compound I (0.46 g, 0.4 mmol), and 10 mL of chlorobenzene (CB) were put into a 100-mL flask under a nitrogen (N₂) atmosphere, the mixture was bubbled with nitrogen for 30 minutes, and then Pd₂(dba)₃ (7.3 mg, 0.008 mmol) and P(o-tol)₃ (9.7 mg, 0.032 mmol) were added thereto, and the resulting mixture was stirred at 110° C. for 72 hours. Thereafter, 0.5 mL of Br-benzotrifluoride was added thereto, and the resulting mixture was stirred at room temperature for 24 hours. The mixed solution was poured into chloroform and allowed to pass through a silica column, and then the solvent was evaporated. Next, after the product was dissolved in chloroform again, the resulting solution was poured into a solution in which 180 mL of methanol and 20 mL of hydrochloric acid at a concentration of 2 M are mixed, and a filtration was performed. The collected polymer was subjected to soxhlet extraction in methanol, acetone, hexane, methylene chloride, and chloroform, and then the chloroform extract was poured into methanol and precipitated. The precipitated polymer was filtered again and dried under a vacuum condition overnight, thereby obtaining Compound 8 (yield: 71%).

### Example 1

A composite solution was prepared by dissolving Compound 1 prepared in the Preparation Example 1 as a donor and PCBM as an acceptor at a ratio of 1:2 in chlorobenzene (CB). In this case, the concentration thereof was adjusted to 2.0 wt %, and the organic solar cell was made to have a structure of ITO/ZnO/a photoactive layer/MoO₃/Ag. A glass substrate coated with ITO was ultrasonically washed using distilled water, acetone, and 2-propanol, and the ITO surface was treated with ozone for 10 minutes, followed by a heat treatment at 120° C. for 10 minutes by spin-coating a ZnO precursor solution. Thereafter, the composite solution was filtered with a 0.45 μm PP syringe filter, and then spin-coated to form a photoactive layer. Thereafter, MoO₃ was deposited onto the photoactive layer to a thickness of 5 nm to 20 nm at a rate of 0.4 Å/s in a thermal evaporator, thereby preparing a hole transport layer. Thereafter, Ag was deposited onto the hole transport layer to a thickness of 10 nm at a rate of 1 Å/s in the thermal evaporator, thereby manufacturing an organic solar cell.

### Example 2

An organic solar cell was manufactured in the same manner as in Example 1, except that Compound 2 was used instead of Compound 1 in Example 1.

### Example 3

An organic solar cell was manufactured in the same manner as in Example 1, except that Compound 3 was used instead of Compound 1 in Example 1.

### Example 4

An organic solar cell was manufactured in the same manner as in Example 1, except that Compound 4 was used instead of Compound 1 in Example 1.

### Example 5

An organic solar cell was manufactured in the same manner as in Example 1, except that Compound 5 was used instead of Compound 1 in Example 1.

### Example 6

An organic solar cell was manufactured in the same manner as in Example 1, except that Compound 6 was used instead of Compound 1 in Example 1.

### Example 7

An organic solar cell was manufactured in the same manner as in Example 1, except that Compound 7 was used instead of Compound 1 in Example 1.

### Example 8

An organic solar cell was manufactured in the same manner as in Example 1, except that Compound 8 was used instead of Compound 1 in Example 1.

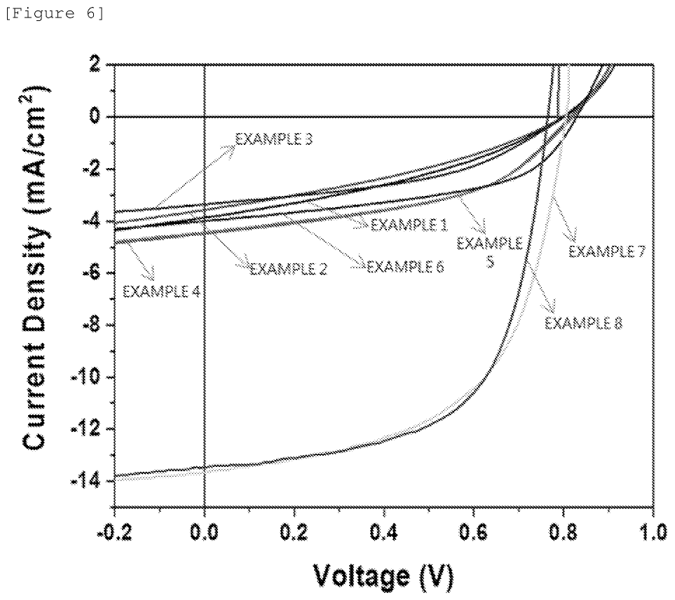

The photoelectric conversion characteristics of the organic solar cells manufactured in Examples 1 to 8 were measured under the condition of 100 mW/cm² (AM 1.5), and the results are shown in the following Table 1 and FIG. 6.

TABLE 1

| | V_{oc} | J_{sc} | | η |
|---|---|---|---|---|
| | (V) | (mA/cm²) | FF | (%) |
| Example 1 | 0.793 | 3.87 | 0.35 | 1.09 |
| Example 2 | 0.801 | 3.57 | 0.34 | 0.97 |
| Example 3 | 0.800 | 3.36 | 0.44 | 1.18 |
| Example 4 | 0.814 | 4.48 | 0.47 | 1.71 |
| Example 5 | 0.815 | 4.36 | 0.47 | 1.67 |
| Example 6 | 0.828 | 4.00 | 0.50 | 1.67 |
| Example 7 | 0.805 | 13.603 | 0.571 | 6.26 |
| Example 8 | 0.765 | 13.461 | 0.618 | 6.36 |

In Table 1, V_{oc}, J_{sc}, FF, and η mean an open-circuit voltage, a short-circuit current, a fill factor, and energy conversion efficiency, respectively. The open-circuit voltage and the short-circuit current are an X axis intercept and a Y axis intercept, respectively, in the fourth quadrant of the voltage-current density curve, and as the two values are increased, the efficiency of the solar cell is preferably increased. In addition, the fill factor is a value obtained by dividing the area of a rectangle, which may be drawn within the curve, by the product of the short-circuit current and the open-circuit voltage. The energy conversion efficiency may be obtained when these three values are divided by the intensity of the irradiated light, and the higher value is preferred.

1. A compound comprising a unit of the following Formula 1: 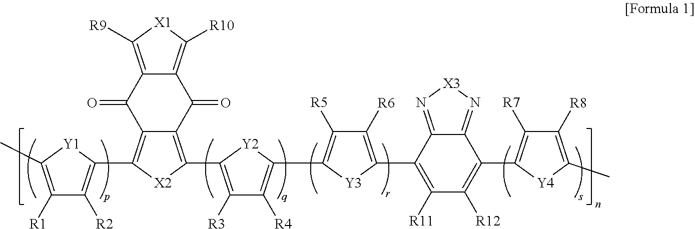
in Formula 1,
p and q are the same as or different from each other, and are each independently an integer from 0 to 3,
when p and q are each 2 or more, each of 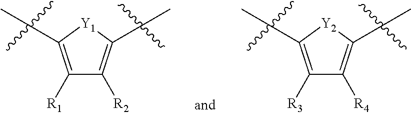
are independently the same as or different from each other,
r and s are the same as or different from each other, and are each independently an integer from 1 to 3,
when r and s are each 2 or more, each of 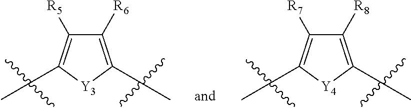
are independently the same as or different from each other,
X1 to X3 are the same as or different from each other, and are each independently S, O, Se, Te, NR, CRR′, SiRR′, PR, or GeRR′,
Y1 to Y4 are the same as or different from each other, and are each independently S, O, Se, Te, NR, CRR′, SiRR′, PR, or GeRR′,
R1 to R12, R, and R′ are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heterocyclic group, and
n is an integer from 1 to 10,000.

2. The compound of claim 1, wherein X1 to X3 are each S.

3. The compound of claim 1, wherein Formula 1 is represented by any one of the following Formulae 1-1 to 1-4: 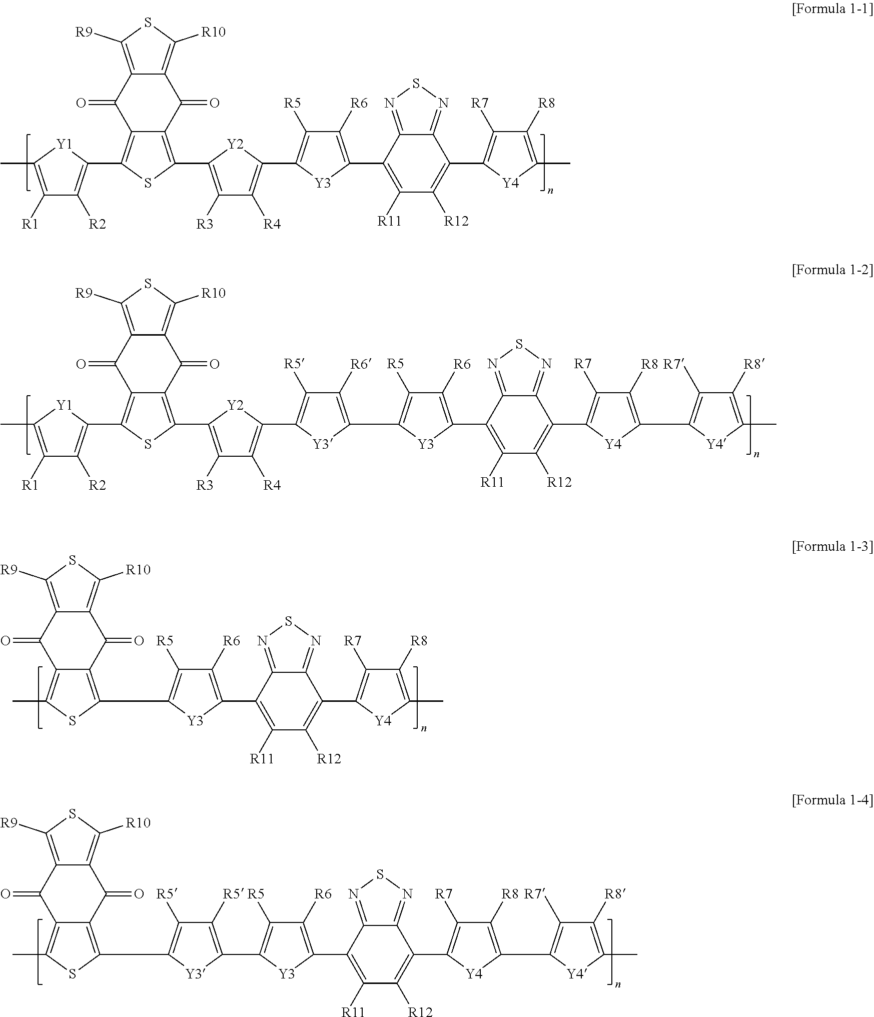
in Formulae 1-1 to 1-4,
n, R1 to R12, and Y1 to Y4 are the same as those defined in Formula 1,
Y3′ and Y4′ are the same as or different from each other, and are each independently S, O, Se, Te, NR, CRR′, SiRR′, PR, or GeRR′,
R5′, R6′, R7′, R8′, R, and R′ are the same as or different from each other, and are each independently hydrogen; deuterium; a halogen group; a nitrile group; a nitro group; a hydroxyl group; a carbonyl group; an ester group; an imide group; an amide group; a substituted or unsubstituted alkyl group; a substituted or unsubstituted cycloalkyl group; a substituted or unsubstituted alkoxy group; a substituted or unsubstituted aryloxy group; a substituted or unsubstituted alkylthioxy group; a substituted or unsubstituted arylthioxy group; a substituted or unsubstituted alkylsulfoxy group; a substituted or unsubstituted arylsulfoxy group; a substituted or unsubstituted alkenyl group; a substituted or unsubstituted silyl group; a substituted or unsubstituted boron group; a substituted or unsubstituted amine group; a substituted or unsubstituted arylphosphine group; a substituted or unsubstituted phosphine oxide group; a substituted or unsubstituted aryl group; or a substituted or unsubstituted heterocyclic group.

4. The compound of claim 1, wherein R1 to R8 are the same as or different from each other, and are each independently hydrogen; a halogen group; a substituted or unsubstituted alkyl group; or a substituted or unsubstituted alkoxy group.

5. The compound of claim 1, wherein R9 and R10 are the same as or different from each other, and are each independently a substituted or unsubstituted alkyl group.

6. The compound of claim 1, wherein R11 and R12 are the same as or different from each other, and are each independently hydrogen; a halogen group; or a substituted or unsubstituted alkoxy group.

7. The compound of claim 1, wherein Y1 to Y4 are each S.

8. The compound of claim 1, wherein p and q are the same as each other, and are each 0 or 1, and
r and s are the same as each other, and are each 1 or 2.

9. The compound of claim 1, wherein Formula 1 is represented by any one of the following compounds: 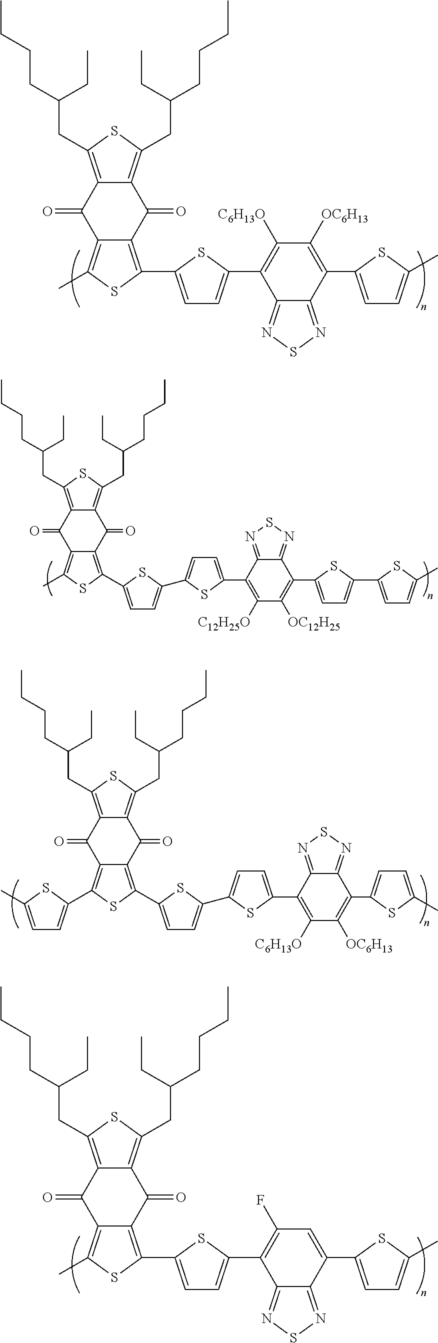 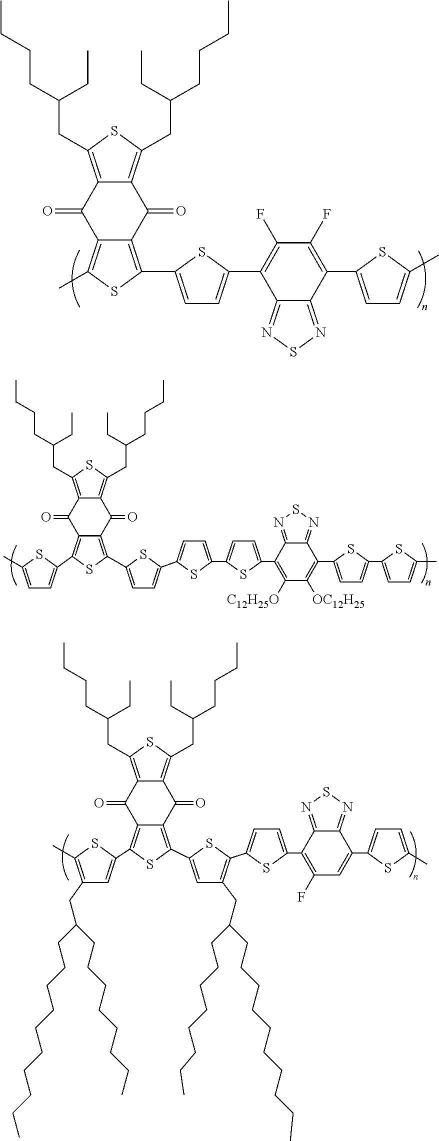 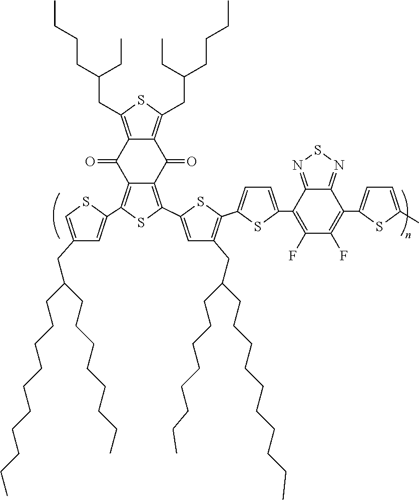
in the compounds, n is an integer from 1 to 10,000.

10. An organic solar cell comprising:
a first electrode;
a second electrode provided to face the first electrode; and
an organic material layer having one or more layers provided between the first electrode and the second electrode and comprising a photoactive layer,
wherein one or more layers of the organic material layer comprise the compound according claim 1.

11. The organic solar cell of claim 10, wherein the organic material layer comprises a hole transport layer, a hole injection layer or a layer which simultaneously transports and injects holes, and
the hole transport layer, the hole injection layer, or the layer which simultaneously transports and injects holes comprises the compound.

12. The organic solar cell of claim 10, wherein the organic material layer comprises an electron injection layer, an electron transport layer or a layer which simultaneously injects and transports electrons, and
the electron injection layer, the electron transport layer or the layer which simultaneously injects and transports electrons comprises the compound.

13. The organic solar cell of claim 10, wherein the photoactive layer comprises an electron donor and an electron acceptor, and
the electron donor comprises the compound.

14. The organic solar cell of claim 10, wherein the organic solar cell further comprises one or two or more organic material layers selected from the group consisting of a hole injection layer, a hole transport layer, a hole blocking layer, a charge generation layer, an electron blocking layer, an electron injection layer, and an electron transport layer.

15. The compound of claim 1, wherein R9 and R10 are 2-ethylhexyl.

16. The compound of claim 1, wherein Formula 1 is represented by any one of the following Formulae 2-1 to 2-4: 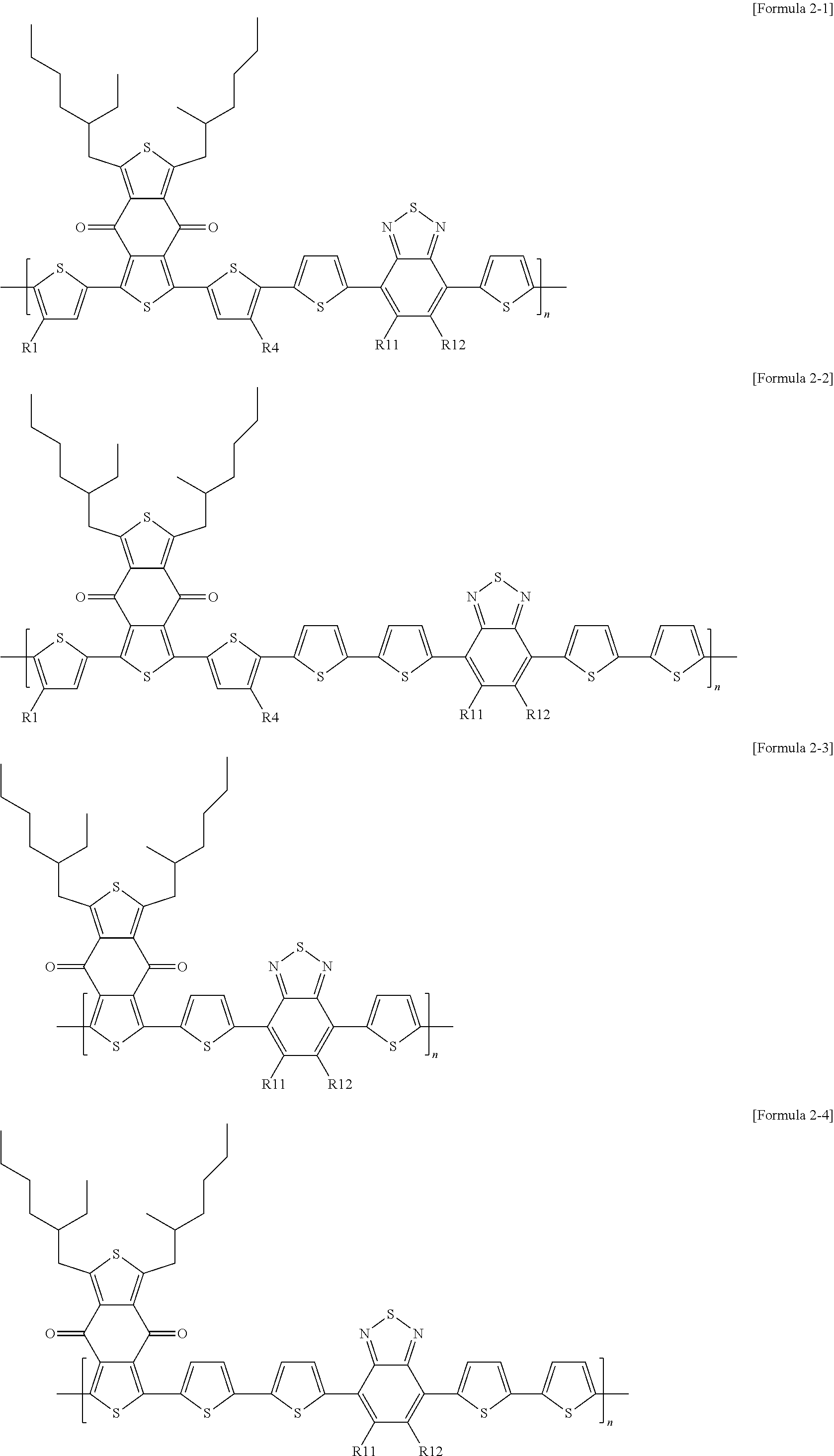
in Formulae 2-1 to 2-4,
R1, R4, R11, R12, and n are the same as those defined in Formula 1.

17. The organic solar cell of claim 13, wherein the electron acceptor comprises at least one selected from the group consisting of fullerene, fullerene derivatives, bathocuproine, semi-conducting elements, semi-conducting compounds, and combinations thereof.

18. The organic solar cell of claim 13, wherein the electron donor and the electron acceptor are present in at a ratio (w/w) of 1:10 to 10:1.

19. The organic solar cell of claim 13, wherein the photoactive layer has a bilayer thin film structure including an n-type organic material layer and a p-type organic material layer, and
the p-type organic material layer includes the compound.
